(19) United States
(12) Patent Application Publication
(10) Pub. No.: US 2004/0254430 A1
(43) Pub. Date: Dec. 16, 2004

(54) Method and apparatus for monitoring an obstetrics patient

(76) Inventor: Emily F. Hamilton, Verdun (CA)

Correspondence Address: LADAS & PARRY LLP, 224 SOUTH MICHIGAN AVENUE, SUITE 1200, CHICAGO, IL, 60604, US

(21) Appl. No.: 10/600,990

(22) Filed: Jun. 13, 2003

(51) Int. Cl. A61B 5/00

(52) U.S. Cl. 600/300; 128/920; 600/588

(57) ABSTRACT

A method and an apparatus for monitoring an obstetrics patient are provided. The apparatus includes an input for receiving a signal indicative of a measurement of a health characteristic of the obstetrics patient, the measurement being associated with a likelihood of a certain outcome. A processing unit processes the signal to derive data indicative of an action for causing a change in the likelihood of the certain outcome. The data indicative of the action for causing the likelihood of the certain outcome to be modified is then released to be conveyed to a user. The data derived by the processing unit is indicative of an action for causing a change to the health characteristic, such as to cause the change to the likelihood of the certain outcome.

### FIELD OF THE INVENTION

The present invention relates generally to the field of obstetrics, and more specifically to a method and apparatus for monitoring the health characteristics of an obstetrics patient.

### BACKGROUND OF THE INVENTION

Devices and machines for monitoring the health characteristics of an obstetrics patient are known in the art. For example, systems for monitoring the heart rate and contraction pattern are commonly used. Measurements are provided either by sensors, as in the case of heart rate, or by manual inputs that are entered by a health care provider, as in the case of measurements of cervical dilation. The systems are able to receive data that are indicative of measurements of a health characteristic being monitored and are able to provide a display that can be viewed by a health care professional. The display can be output via a printout, or via a display screen, for example.

A deficiency with existing systems is that they are generally unable to inform a health care professional as to what the measurements actually mean. For example, the systems are unable to provide any indication as to whether the measurement readings mean that the patient is on the right track or whether the patient is at risk. Therefore, the health care professional must rely solely on his or her judgement in order to decide on the best treatment for the patient. This often results in liability issues for the hospital and a quality of care that is not uniform for all patients. This results in the patients having to depend entirely on the knowledge of the health care professional attending to their needs.

Therefore, in the context of the above, there is a need in the industry to provide a method and apparatus for monitoring the health characteristics of an obstetrics patient that alleviates at least in part problems associated with the existing methods and devices.

### SUMMARY OF THE INVENTION

In accordance with a first broad aspect, the present invention provides an apparatus for monitoring an obstetrics patient. The apparatus comprises an input, a processing apparatus and an output. The input is adapted to receive a signal that is indicative of a measurement of a health characteristic of the obstetrics patient. The measurement of the health characteristic is associated with a likelihood of a certain outcome. The processing unit is coupled to the input and is operative for processing the signal in order to derive data indicative of an action for causing a change in the likelihood of the certain outcome. Finally, the output is adapted to release the data indicative of the action for the likelihood of the certain outcome to be modified.

In a specific example of implementation, the data derived by the processing unit is indicative of an action for causing the health characteristic to be modified.

In a further specific example of implementation, the measurement of the health characteristic of the obstetrics patient is indicative of a measurement of a health characteristic of a fetus.

In a further specific example of implementation, the measurement of the health characteristic of the obstetrics patient is indicative of a measurement of a health characteristic of a pregnant woman.

In a still further specific example of implementation, the processing unit is adapted to process the signal indicative of a measurement of a health characteristic in order to derive a data element indicative of the likelihood of the certain outcome and for deriving data indicative of an action for causing the health characteristic to be modified. The data indicative of an action for causing the health characteristic to be modified is derived at least in part on the basis of the data element indicative of the likelihood of the certain outcome and the signal indicative of the measurement of the health characteristic.

In accordance with another broad aspect, the present invention provides a method for monitoring an obstetrics patient. The method comprises receiving a signal indicative of a measurement of a health characteristic of the obstetrics patient, the measurement being associated with a likelihood of a certain outcome. The method further involves, processing the signal to derive data indicative of an action for causing a change in the likelihood of the certain outcome. And finally, the third step involves releasing the data indicative of the action for causing the likelihood of the certain outcome to be modified.

In accordance with yet another broad aspect, the present invention provides a computer readable storage medium including a program element suitable for execution by a computing apparatus for monitoring the health characteristics of an obstetrics patient. The computing apparatus has a memory unit and a processor operatively connected to the memory unit for monitoring the health characteristics of an obstetrics patient in accordance with the above-described method.

In accordance with yet another broad aspect, the present invention provides a system for monitoring an obstetrics patient. The system comprises an interface for receiving a measurement of a health characteristic of the obstetrics patient, the measurement being associated with a likelihood of a certain outcome. The system further comprises an apparatus having an input for receiving a signal indicative of the measurement of the health characteristic of the obstetrics patient, a processing unit coupled to the input, and an output coupled to the processing unit. The processing unit is operative for processing the signal indicative of the measurement of the health characteristic to derive data indicative of an action for causing a change in the likelihood of the certain outcome. The output is suitable for releasing an output signal for causing a display unit to display information derived on the basis of the data indicative of the action for causing the likelihood of the certain outcome to be modified. The system also comprises a display unit coupled to the output of the apparatus, the display unit being responsive to the output signal to display the information derived on the basis of the data indicative of the action for causing the likelihood of the certain outcome to be modified.

In accordance with yet another broad aspect, the present invention provides a server system for monitoring an obstetrics patient, the server system storing a program element for execution by a CPU. The program element comprises a first program element component for receiving a measurement of a health characteristic of the obstetrics patient. The measurement is associated with a likelihood of a certain outcome. The program element also includes a second program element component for processing the measurement to derive data indicative of an action for causing a change in the likelihood of the certain outcome. The program element also includes a third program element component for causing the data indicative of the action to be conveyed to a user.

In accordance with yet another broad aspect, the present invention provides a client-server system for monitoring an obstetrics patient. The client-server system includes a client system and a server system operative to exchange messages over a data network. The server system stores a program element for execution by a CPU. The program element comprises a first program element component for execution on the server system for receiving a measurement of a health characteristic of the obstetrics patient. The measurement is associated with a likelihood of a certain outcome. The program element also comprises a second program element component for execution on the server system for processing the measurement to derive data indicative of an action for causing a change in the likelihood of the certain outcome. The program element also comprises a third program element component for execution on the server system for sending messages to the client system for causing the client system to display information on the basis of the data indicative of the action for causing the likelihood of the certain outcome to be modified. The program element also includes a fourth program element component for execution on the client system for receiving a message from the server system for conveying the information to a user.

These and other aspects and features of the present invention will now become apparent to those of ordinary skill in the art upon review of the following description of specific embodiments of the invention in conjunction with the accompanying drawings.

### BRIEF DESCRIPTION OF THE DRAWINGS

In the accompanying drawings:

FIG. 1 shows a high-level functional block diagram of an apparatus for monitoring the health characteristics of an obstetrics patient in accordance with a specific example of implementation of the present invention;

FIG. 2 shows a flow diagram of a method for monitoring the health characteristics of an obstetrics patient in accordance with a specific example of implementation of the present invention;

FIG. 3 shows a non-limiting example of a visual representation of the data released by the processing unit shown in FIG. 1 for monitoring the health characteristics of an obstetrics patient in accordance with a specific example of implementation of the present invention;

FIG. 4 shows a computing unit for implementing an apparatus for monitoring an obstetrics patient in accordance with a specific embodiment of the present invention;

FIG. 5 shows a functional block diagram of a client-server system for monitoring an obstetrics patient in accordance with an alternative specific non-limiting example of implementation of the present invention;

FIG. 6 is a high level conceptual block diagram of a program element suitable for execution on the client server system shown in FIG. 5 in accordance with a first specific example of implementation of the present invention; and

FIG. 7 is a high level conceptual block diagram of a program element suitable for execution on the element server system shown in FIG. 5 in accordance with a second specific example of implementation of the present invention.

Other aspects and features of the present invention will become apparent to those ordinarily skilled in the art upon review of the following description of specific embodiments of the invention in conjunction with the accompanying figures.

### DETAILED DESCRIPTION

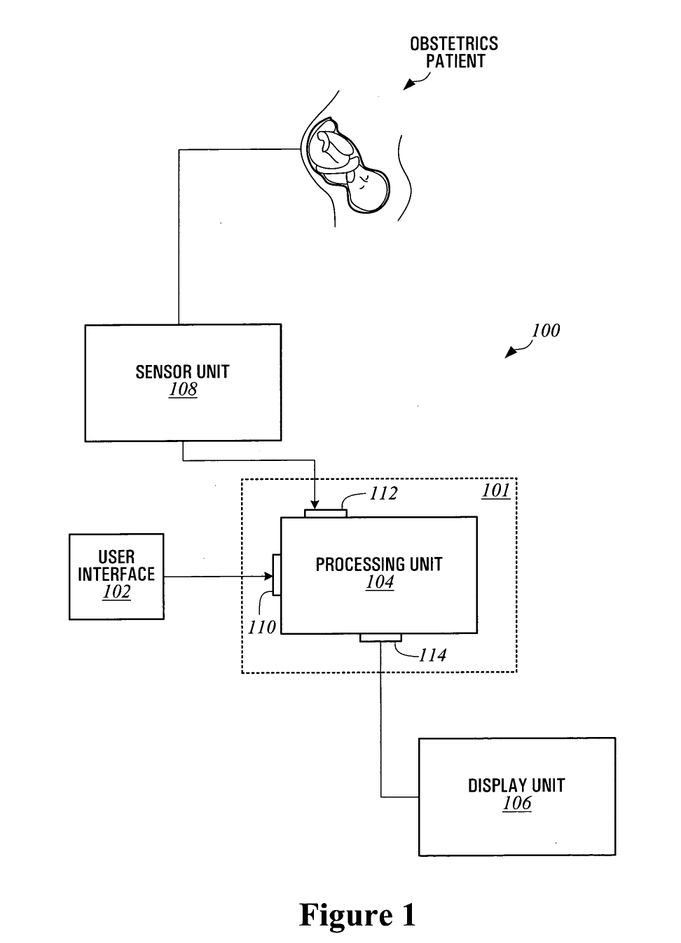

With reference to FIG. 1, there is shown a configuration of a system 100 for monitoring the health characteristics of an obstetrics patient. As used herein, the term “obstetrics patient” refers to a pregnant woman, a fetus in the womb or a newborn baby.

As shown in FIG. 1, the system 100 comprises a user interface 102, a sensor unit 108, an apparatus 101 containing a processing unit 104, and a display unit 106. In an alternative embodiment, system 100 does not comprise a sensor unit 108, and in yet another alternative embodiment, system 100 includes multiple sensor units 108 to provide different measurements.

The user interface 102 includes any one or a combination of a keyboard, a pointing device, a touch sensitive surface or a speech recognition unit. The user interface 102 enables a user to enter health information relating to a certain obstetrics patient. The information can include measurements relating to one or more health characteristics, and/or information relating to the patient's medical history. Examples of non-limiting health characteristics include heart rate, blood pressure, blood type, body temperature, cervical ripeness score, Human Immunodeficiency virus (HIV) status, Hepatitis B status, Group B streptococcal status, Rubella status, and prenatally detected pylectasis, among others.

The cervical ripeness score is a semi-quantitative measure of the compliance of the cervix. More specifically, the score is an indication of the softening, shortening and dilation of the cervix during labour. Any type of calculation of the cervical ripeness score can be used, such as the Bishop's Score, which is based on five commonly measured features of the pelvic exam, namely cervical dilation (measured in cm), effacement (measured as a %), the station of the fetus above or below the ischial spines (measured in cm), the position of the cervix with respect to its axis in the vagina and the consistency of the vagina (from soft to firm). For more information relating to Bishop's Score, the reader can reference Bishop E. H. (1964) Pelvic Scoring for Elective Induction. Obstet Gynecol 24, 266. The content of this document is incorporated herein by reference.

The information relating to the patient's medical history can include any diseases the patient might have, such as diabetes or HIV/AIDS, and any information regarding the patient's previous pregnancies, such as a premature baby, a previous cesarean section or current infectious disease status or blood group.

In addition to the user interface 102, system 100 includes a sensor unit 108 that is capable of obtaining measurements of one or more health characteristics of an obstetrics patient. The sensor unit 108 can include one or more measurement devices. For example, the sensor unit 108 can include a heart rate monitor, a blood pressure monitor and a body temperature monitor, among other possible measurement devices. Therefore, instead of having a user manually enter measurements of one or more health characteristics via user interface 102, sensor unit 108, is operable to automatically take measurements of one or more health characteristics, and transmit signals indicative of those measurements directly to the processing unit 104. In addition, the sensor unit 108 has the advantage that it is able to automatically provide the processing unit 104 with a plurality of measurements at spaced intervals in time. This is useful for health characteristics such as heart rate that ideally should be monitored with respect to time.

Measurements that are semi-quantitative, such as the cervical ripeness score, can be calculated manually by the health care professional, and entered into processing unit 104 via user interface 102, or alternatively, the health care professional can allow the processing unit 104 to do the calculations based on certain measurements. For example, for the semi-quantitative measurement of a Bishop's score, the health care professional can take the necessary five measurements of cervical dilation, effacement, the station of the fetus above or below the ischial spine, the position and consistency of the cervix, and use those measurements to manually calculate the patient's Bishop's score. Once the Bishop's score has been manually calculated, the health care professional can then enter that score into processing unit 104 via user interface 102. Alternatively, the health care professional can provide the processing unit 104 with the five measurements needed to calculate the Bishop's score, and allow the processing unit 104 to compute the Bishop's score based on those five measurements. In such an implementation, the processing unit 104 includes the necessary algorithms in order to derive the semi-quantitative measurement.

In the specific embodiment shown in FIG. 1, apparatus 101 includes a processing unit 104, two inputs 110 and 112, and an output 114. Input 110 is operative for receiving signals from the user interface 102 indicative of information about the obstetrics patient. As mentioned above, this information can include measurements relating to one or more health characteristics, as well as information regarding the patient's medical records. Input 112 is operative for receiving signals from sensor unit 108 that are indicative of health characteristics of the obstetrics patient.

The measurements relating to the one or more health characteristics are associated with a likelihood of a certain outcome. Table 1, shown below, provides a list of health characteristics with associated outcomes. In addition, Table 1 provides a suggested intervention for decreasing the likelihood of the certain outcome. The use of the suggested intervention, or recommended action, will be described in more detail further on in the specification.

TABLE 1

| Health | | |
|---|---|---|
| Characteristic | Explanation of Associated Likelihood | Possible Intervention |
| Cervical | The cervical score is a semi-quantitative measure | Prior to inducing labor, medications |
| scoring | of the compliance of the cervix. Lower scores | and/or mechanical methods may be |
| | indicate less compliance than higher scores. | used to improve the score and |
| | Lower scores on admission are associated with | therefore improve the likelihood of |
| | higher likelihood of cesarean section | vaginal delivery |
| Group B | Group B streptococcal colonization of the | Administration of antibiotics to the |
| streptococcal | maternal urogenital tract carries increased risk of | mother for several hours (at least 4) |
| status | fetal and newborn infection with this bacteria. | before birth can reduce this risk for |
| | Newborn infection can lead to serious illness | the baby. |
| | including sepsis and death. In the absence of | |
| | recent tests for the bacteria, certain clinical | |
| | conditions indicative of potential infection also | |
| | warrant prophylactic treatment. | |
| HIV status | Positive HIV status in the mother carries specific | Treatment with antivirals and |
| | risk that HIV will be transmitted to the baby. | delivery by scheduled cesarean |
| | | section before labor or rupture of |
| | | the membranes reduce the |
| | | likelihood of the baby contracting |
| | | HIV at the time of delivery. |
| | | Recommendations can range from |
| | | the use of specific medications, to |
| | | birth by cesarean as well as |
| | | supportive health care services. |
| Hepatitis B | Babies born of mothers carrying this virus are at | A vaccination program instituted |
| status | increased risk of acquiring this infection. | shortly after birth reduces this risk. |
| Rhesus | Mothers who are Rhesus negative and who have | If the baby is Rh positive, |
| negative | no natural antibodies to their baby's Rh positive | administration of Rh immune |
| mother | antigens may become exposed to this antigen at | globulin to unsensitized, Rh |
| | birth. Development of Rh antibodies will produce | negative mothers after birth will |
| | increased risk of fetal anemia in subsequent | protect them from becoming |
| | pregnancies with Rh positive fetuses. | sensitized. |
| Prenatally- | Increased levels of widening of the real pelvis of | Interventions can range from |
| detected | the fetal ureter carries increased of infection and | postnatal investigation to |
| pylectasis | warrants postnatal followup to determine its | prophylactic antibiotics to |
| | course and etiology and treatment. | immediate urological investigation. |
| Rubella status | Maternal Rubella infection during early | Vaccination of non-immune |
| | pregnancy carries increased risk of fetal rubella | mothers in the postpartum period |
| | infection and sequelae. | will reduce the risk of fetal rubella |
| | | infection in subsequent pregnancies. |

It will be appreciated that the above described list of health characteristics is not exhaustive and has been presented for the purpose of illustration only. In addition, the “possible interventions” associated to the various health characteristics may differ without detracting from the spirit of the invention.

A more detailed example of the risks associated with cervical scoring (i.e., cervical ripeness) will now be described. For example, it is known in gynecological fields that the ripeness of a woman's cervix on admission to the hospital is associated with the likelihood that a cesarean section will be required. As such, the cervical ripeness score has a high correlation with the rate of cesarean section. A ripe cervix is more likely to dilate when a force is applied against it by the uterine contractions, whereas an unripe cervix is less likely to dilate. As such, an unripe cervix may require many hours of uterine contractions before it begins to dilate. Slow progress during labour is one of the main causes of cesarean section. Therefore, a woman having a low cervical ripeness score is far more likely to have a cesarean section than a woman with a high cervical ripeness score.

As shown in FIG. 1, the processing unit 104 is coupled to inputs 110 and 112 and is operative for processing the signal or signals indicative of measurements of one or more health characteristics received at inputs 110 and 112. As will be described in more detail below, on the basis of the signal or signals received, the processing unit 104 is operative to derive data indicative of an action for causing one or more of the health characteristics to be modified, which in turn results in a change in the likelihood of the certain outcome. For example, in the specific case where the health characteristic being monitored is the cervical ripeness score, and the patient's cervical ripeness score is low, the processing unit will derive data indicative of an action that can be taken by a health care professional in order to increase the cervical ripeness score. For example, the action could be a treatment for affecting the ripeness of the cervix using physical devices and/or pharmacological agents. These actions can reduce the likelihood that the patient will have to undergo a cesarean section.

In a specific example of implementation, the processing unit 104 is adapted for processing the signal or signals received at input 110 and/or input 112, in order to derive a data element indicative of the likelihood of a certain outcome. The certain outcome is generally an undesirable outcome, thereby providing an early warning to health care professionals that the patient is at risk. For example, in the case where the measurement of the patient's health characteristic is the cervical ripeness score, the processing unit 104 processes the signal in order to derive another score that is indicative of the likelihood that the patient will give birth by cesarean section. As a non-limiting example, the score could be indicated in terms of the probability that the patient will have to undergo a cesarean section.

In the cases where the processing unit 104 derives a data element indicative of the likelihood of a certain outcome, the data indicative of an action for causing the measurement of the health characteristic to be modified, which in turn causes the likelihood of the certain outcome to be modified, is derived at least in part on the basis of the data element indicative of the likelihood of the certain outcome and the signal indicative of the measurement of the health characteristic.

In an alternative example of implementation, the step of deriving a data element indicative of the likelihood of a certain outcome is omitted. In such an alternative implementation, the associated likelihood is inherent given the measurements of the patient's health characteristic. In such cases, the processing unit 104 is operative to derive an action for causing the measurement of the patient's health characteristic to be modified without first deriving a data element indicative of a likelihood of a certain outcome.

In a specific example of implementation, the processing unit 104 uses a database in order to derive the data indicative of an action for causing the measurement of the patient's health characteristic to be modified. For example, in the case where the health characteristic being monitored is a patient's cervical score, the cervical score is received at either input 110 or 112, and the processing unit 104 is adapted to look up an action associated to that input from a database of the type shown in Table 2, below. The processing unit 104 then outputs the “associated action” to a display unit, for viewing by a health care professional.

TABLE 2

| Cervical Score | Associated Action |
|---|---|
| 0-20% | In your institution, the rate of cesarean associated with this score is high and ranges |
| | from 40 to 53%. To lessen this likelihood, consider administering a cervical |
| | ripening agent and then reevaluating the cervical score and indication for induction. |
| | Consider also deferring the induction if medically desirable. |
| 21-40% | In your institution, the rate of cesarean associated with this score is in an |
| | intermediate level and ranges from 27 to 40%. To lessen this likelihood, consider |
| | administering a cervical ripening agent and then reevaluating the cervical score and |
| | indication for induction. Consider deferring induction if medically feasible. |
| 41-60% | In your institution, the rate of cesarean associated with this score is in a low level |
| | and ranges from 14% to 27%. Some improvements to lessen this likelihood will |
| | occur with use of cervical ripening agents. |
| Over 60% | In your institution, the rate of cesarean associated with this score is at the lowest |
| | level and ranges from 0 to 14%. There is no significant benefit with the additional |
| | use of cervical ripening agents. |

Table 2 is a specific, non-limiting example of a database that can be accessed by processing unit 104 when the health characteristic being monitored is the cervical ripeness score. This health characteristic is correlated with a likelihood that the patient will have to deliver by cesarean. The decision to induce labor is based upon clinical judgment concluding that earlier delivery with its concomitant risks and benefits outweighs the associated risks and benefits of the continuation of the pregnancy. One of the risks of induction is a higher rate of cesarean section. This risk is largely determined by the state of cervical compliance at the onset of the induction. Use of pharmacological or mechanical agents can ripen the cervix and reduce this risk.

Referring back to Table 2, in order to derive an action for causing the measurements of the health characteristic to be modified, the processing unit 104 compares the measurement of the patient's health characteristic received at the input 110 to the measurements, or ranges of measurements, contained within the database. As shown in Table 2, each measurement (or range of measurements) is mapped to an associated action. It should be understood that the scores and actions provided in Table 2, above, are strictly for illustrative purposes, and may not be the exact scores or actions that would be included in an actual database.

As shown in Table 2, the database contains a threshold measurement, above which the associated action is “there is no significant benefit with the additional use of cervical ripening agents”. In other words, the threshold measurement or range of measurements separate the measurements that are not associated with an action, from those that are associated with an action. In the case of the cervical ripeness scores contained above, the threshold range is over 60%. However, for cervical ripeness scores that fall below that range, the associated action is to provide the patient with medication for causing a change in the measurement of the cervical ripeness score. It should be understood that different actions can be associated with different measurement scores. For instance, the type of cervical ripening agent can be altered, depending on the measurement scores. Some measurement scores might call for a stronger cervical ripening agent than others.

Although the database illustrated in Table 2 shows that the associated actions are mapped to measurements of health characteristics, it should be understood that in an alternative example of implementation, the associated actions can be mapped to data elements that are indicative of the likelihood of the certain outcome, where the likelihood of the certain outcome is associated with a certain measurement of a health characteristic. For example, in the instance where the likelihood of the certain outcome is a percentage risk of giving birth by cesarean section, a first action could be associated with a 40% risk of giving birth by cesarean section, and a second action is associated with a 20% risk of giving birth by cesarean section.

In another example of implementation, in the case where the health characteristic being monitored is whether or not a patient with HIV/AIDS is in labor and whether or not there has been membrane rupturing, the processing unit 104 would “look up” an associated action from a database of the type shown in Table 3, below. In such a scenario, the data received at input 110 would be whether the patient with HIV/AIDS is in labor and whether there has been a rupture of the amniotic membranes. Based on these inputs, the processing unit 104 is operative to access a database such as the specific, non-limiting database shown in Table 3, in order to look up an action associated to those inputs, that provides a suggestion as to how to reduce the likelihood that a baby born to that patient would contract HIV/AIDS. The processing unit 104 is then operative to output that action to a display unit.

TABLE 3

| | Has there | |
|---|---|---|
| | been a | |
| Is the | recent | |
| HIV/AIDS | rupture | |
| patient | of the | |
| in labor | membranes | Associated Action |
| YES | YES | Commence antiretroviral medication. Perform |
| | | cesarean section without undue delay as |
| | | duration of rupture of membranes and/or |
| | | vaginal delivery increase risk of |
| | | HIV transmission to the baby. |
| | NO | Commence antiretroviral medication. Perform |
| | | elective cesarean section after 3 hours of |
| | | infusion or sooner to avoid vaginal delivery |
| | | or prolonged active labor. |
| NO | YES | Commence antiretroviral medication infusion. |
| | | Perform cesarean section without undue |
| | | delay as longer duration of membrane |
| | | rupture diminishes the benefit of |
| | | cesarean birth. |
| | NO | Commence antiretroviral medication infusion. |
| | | Perform elective cesarean section |
| | | after 3 hours of infusion. |

Transmission of HIV from mother to newborn can occur at the time of birth. As such, in the case where the patient has HIV/AIDS there is a high likelihood that a baby born to that patient will also develop HIV/AIDS. Cesarean section should not be used indiscriminately because the surgery and/or recovery can be very complicated in a mother who has immune depression from HIV/AIDS. It should be reserved for situations where on balance there are more benefits expected for the mother and baby compared to the concomitant complications. Therefore, in such a scenario, the system 100 is used in order to provide the health care professional with the necessary procedures and actions to follow in order to reduce the likelihood that the baby will contract HIV/AIDS. For more information, the reader is invited to reference The International Perinatal HIV Group Engle J. N. (1999) The mode of delivery and the risk of vertical transmission of human immunodeficiency virus type 1: a meta-analysis of 15 prospective cohort studies. 340:977-987, which is incorporated herein by reference.

The baby can contract HIV/AIDS through transplacental mixing of maternal and fetal blood when the usual barriers to this mixing are more subject to disruption, such as during contractions or the birth process. In addition, the fetus is more exposed to cervicovaginal secretions and maternal blood during labor or periods of membrane rupture or vaginal birth. Lower maternal viral level and use of antiretroviral medications prenatally, intrapartum and in the early newborn period further decrease the likelihood of viral transmission to the baby. For more information, the reader is invited to reference Connor E M, Sperling R S, Gelber R, Kiselev P, Scott G, O'Sullivan M J et al. Pediatric AIDS Clinical Trials Group Protocol 076 Study Group (1994) Reduction of maternal-infant transmission of human immunodeficiency virus Type 1 with Zidoviduine treatment. 331:1173-1180, which is incorporated herein by reference.

It should be understood that the “associated actions” contained in the Tables 1, 2 and 3 represent current best practices based on available evidence to date. As clinical advances occur, appropriate treatments and “associated actions” will change. In addition, the purpose of the “associated actions” are for illustration purposes only, and are not a limiting feature of the invention.

Referring back to FIG. 1, once the processing unit 104 has derived the data indicative of an action for causing one or more of the health characteristics to be modified, output 114 releases the data derived by processing unit 104 to a display unit 106. The display unit 106 is coupled to the output 114 of apparatus 101 and is responsive to the released signal for displaying the information derived by processing unit 104. The display unit 106 may be in the form of any suitable device for conveying to the physician or other health care professional the data indicative of the action for causing the health characteristic to be modified. In a specific example of implementation, the display unit 106 can include a display screen, or in an alternative example of implementation, the display unit 106 can include a printing device for displaying the data in printed form on paper.

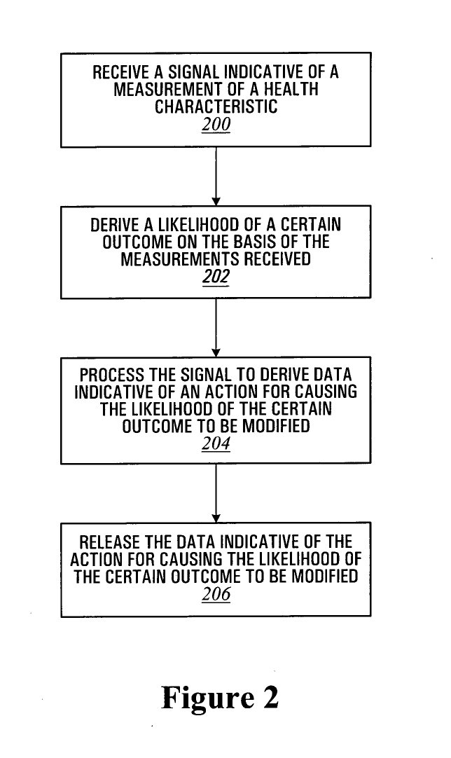

The method for monitoring an obstetrics patient as implemented by apparatus 101 is described in greater detail herein below with reference to the flow chart shown in FIG. 2.

At step 200 the apparatus 101 receives a signal indicative of a measurement of a certain health characteristic. As explained above, the measurement can be received from data entered at a user interface 102, or alternatively can be obtained by a sensor unit 108 that automatically obtains measurements from a patient. At step 202, the measurement is associated with a likelihood of a certain outcome, which, as mentioned above, could be derived by the processing unit 104 or could be an inherent outcome given the measurements of the patient's health characteristic. At step 204, the processing unit 104 compares the likelihood of the certain outcome, or the measurement of the patient's health characteristic to corresponding values, contained in a database, that are mapped to specific actions. In this way, the processing unit 104 is able to derive data indicative of an action for causing a reduction in the likelihood of the certain outcome or a change in the health characteristics measurements, at least in part on the basis of the signals indicative of measurements of the patient's health characteristics. Finally, at step 206, the data indicative of the action is released to the user. Preferably, this data is released to a display unit 106 such that the action is displayed to a health care professional as described above.

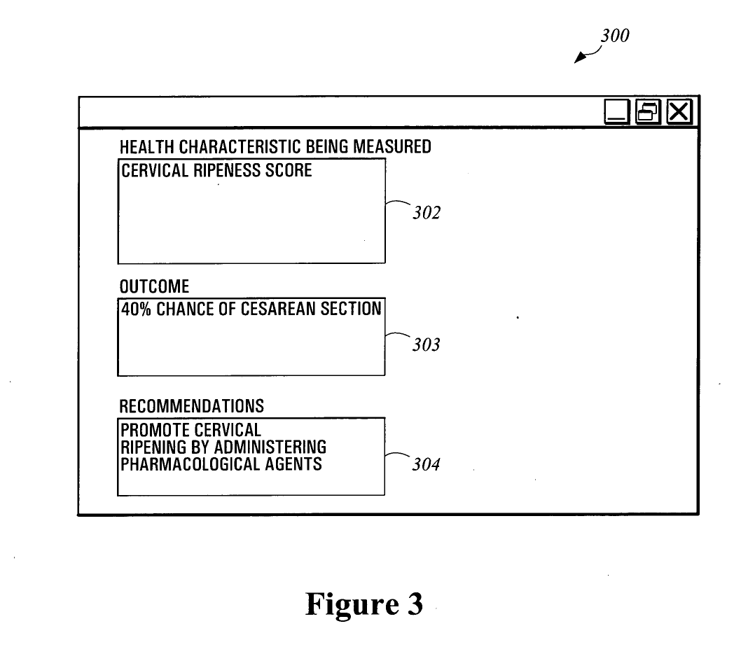

A non-limiting example of a visual representation of the data released by processing unit 106 is shown in FIG. 3. The visual representation is in the form of a window 300 that could be shown on a computer display screen. The window contains three data fields 302, 303 and 304. Data field 302 is a text box that identifies the health characteristic being measured, as well as measurements of that health characteristic. In the specific example shown, the health characteristic is the cervical ripeness score. Data field 303 is a text box that indicates the outcome associated with the measurements of the health characteristic. In the specific example shown, the outcome is a 40% chance of having to have a cesarean section. Data field 304 is a text box that displays the data indicative of one or more actions for causing the health characteristic or the certain outcome to be modified.

It should be understood that window 300, as shown in FIG. 3, is only an example of one specific visual representation of how the data derived by processing unit 104 can be displayed. It is within the scope of the invention for the visual representation to contain more or less information. For example, the display unit 106 could display the actual measurements of the health characteristic being measured. As such, the health care professional could see how the measurements of the health characteristic and the likelihood of the certain outcome change when the recommended action is implemented. It should also be understood that multiple health characteristics could be measured and displayed or, alternatively, only the relevant information being monitored could be displayed.

Those skilled in the art should appreciate that in some embodiments of the invention, all or part of the functionality for monitoring an obstetrics patient, previously described herein with respect to the apparatus 101, may be implemented as pre-programmed hardware or firmware elements (e.g., application specific integrated circuits (ASICs), electrically erasable programmable read-only memories (EEPROMs), etc.) or other related components.

In other embodiments of the invention, all or part of the functionality previously described herein with respect to the apparatus 101 for monitoring an obstetrics patient may be implemented as software consisting of a series of instructions for execution by a computing unit. The series of instructions could be stored on a medium which is fixed, tangible and readable directly by the computing unit (e.g., removable diskette, CD-ROM, ROM, PROM, EEPROM or fixed disk) or the instructions could be stored remotely but transmittable to the computing unit via a modem or other interface device (e.g., a communications adapter) connected to a network over a transmission medium. The transmission medium may be either a tangible medium (e.g., optical or analog communications lines) or a medium implemented using wireless techniques (e.g., microwave, infrared or other transmission schemes).

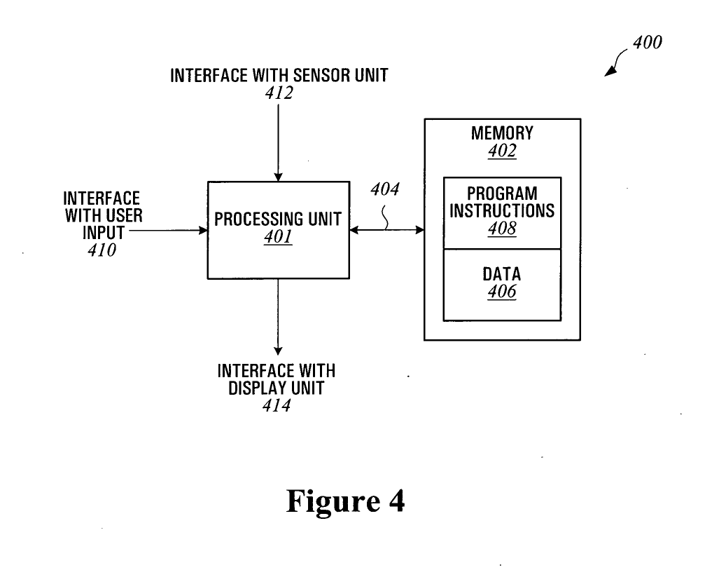

The apparatus 101 for monitoring an obstetrics patient may be configured as a computing unit 400 of the type depicted in FIG. 4, including a processing unit 401 and a memory 402 connected by a communication bus 404. The memory 402 includes data 406 and program instructions 408. The processing unit 401 is adapted to process the data 406 and the program instructions 408 in order to implement the method described in the specification and depicted in the drawings. The computing unit 400 may also comprise a number of interfaces 410, 412 and 414 for receiving or sending data elements to external devices. For example, interfaces 410 and 412 receive signals from user interface 102 and sensor unit 108 as described with respect to FIG. 1, and as such are used for receiving data streams indicative of measurements of one or more health characteristics, wherein the measurements are associated to certain outcome. The processing unit 401 is operative for processing the received signal or signals to derive data indicative of an action for causing a change in the certain outcome. Interface 414 is for releasing the data indicative of an action for causing the likelihood of the certain outcome to be modified. The released data is transmitted to display unit 106, such that display unit 106 conveys the data derived by processing unit 401 to a health care professional.

In a specific example of implementation, the memory 402 includes a program element contained within the program instructions 408, for execution by the computing unit 400. Once the processing unit 401 has derived the data element indicative of an action for causing the likelihood of the certain outcome to be modified, the program element is operative to process the data element so as to be able to covey information to a user on a display unit. As described above, the display unit can be either one of a display screen or a paper printout.

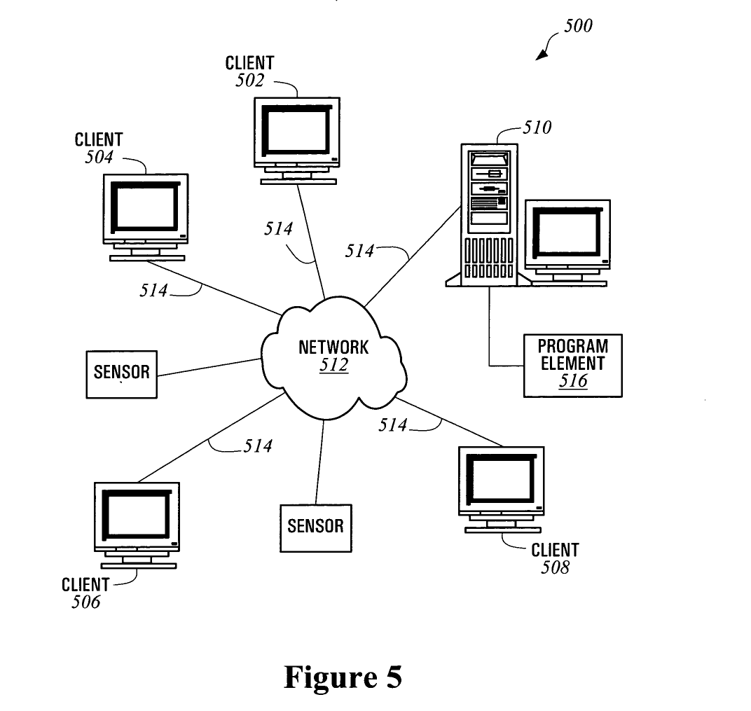

It will be appreciated that the system for monitoring an obstetrics patient may also be of a distributed nature where the measurements of one or more health characteristics are collected at one location and transmitted over a network to a server unit implementing the method as described above. The server unit may then transmit a signal for causing a display unit to convey information to the user. The display unit may be located in the same location where the measurements are being obtained or in the same location as the server unit or in yet another location. FIG. 5 illustrates a network-based client-server system 500 for monitoring the health characteristics of one or more obstetrics patients. The client-server system 500 includes a plurality of client systems 502, 504, 506 and 508 connected to a server system 510 through network 512. The communication links 514 between the client systems 502, 504, 506 and 508 and the server system 510 can be metallic conductors, optical fibres or wireless, without departing from the spirit of the invention. The network 512 may be any suitable network, including but not limited to a global public network such as the Intranet, a private network or a wireless network.

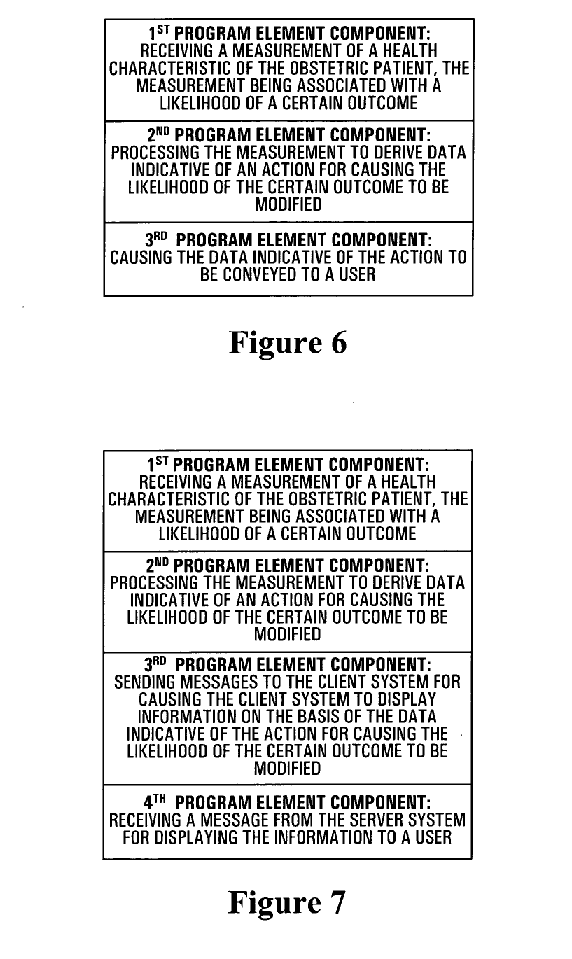

The server 510 may be adapted to process and issue signals concurrently using suitable methods known in the computer related arts. The server system 510 includes a program element 516 for execution by a CPU. Program element 516 implements similar functionality as program instructions 408 (shown in FIG. 4) and includes the necessary networking functionality to allow the server system 510 to communicate with the client systems 502, 504, 506 and 508 over network 512. In a non-limiting example of implementation, program element 516 includes a number of program element components, each program element component implementing a respective portion of the functionality of apparatus 101. FIG. 6 shows a non-limiting example of the architecture of program element 516 at the server system. As shown, the program element 516 includes 3 program element components:

1. The first program element component 600 is for receiving a measurement of a health characteristic of the obstetrics patient, this measurement being associated with a likelihood of a certain outcome.

2. The second program element component 602 is for processing the measurement to derive data indicative of an action for causing a change in the likelihood of the certain outcome.

3. The third program element component 604 is for causing the data indicative of the action to be conveyed to a user.

In an alternative non-limiting example of implementation, program element 516 includes a 4 program element components. FIG. 7 shows a non-limiting example of the architecture of the alternative example of implementation of program element 516 at the server system.

1. The first program element component 700 is executed on server system 510 and is for receiving a measurement of a health characteristic of the obstetrics patient, this measurement being associated with a likelihood of a certain outcome.

2. The second program element component 702 is executed on server system 510 and is for processing the measurement to derive data indicative of an action for causing a change in the likelihood of the certain outcome.

3. The third program element component 704 is executed on server system 510 and is for sending messages to said client system for causing the client system to display information on the basis of the data indicative of the action for causing the likelihood of the certain outcome to be modified.

4. The fourth program element component 706 is executed on server system 510 and is for receiving a message from the server system for displaying the information to a user.

Those skilled in the art should further appreciate that the program instructions may be written in a number of programming languages for use with many computer architectures or operating systems. For example, some embodiments may be implemented in a procedural programming language (e.g., “C”) or an object oriented programming language (e.g., “C++” or “JAVA”).

Although the present invention has been described in considerable detail with reference to certain preferred embodiments thereof, variations and refinements are possible without departing from the spirit of the invention. Therefore, the scope of the invention should be limited only by the appended claims and their equivalents.

1. An apparatus for monitoring an obstetrics patient, said apparatus comprising:
a) an input for receiving a signal indicative of a measurement of a health characteristic of the obstetrics patient, the measurement being associated with a likelihood of a certain outcome;
b) a processing unit coupled to said input, said processing unit being operative for processing said signal to derive data indicative of an action for causing a change in the likelihood of the certain outcome;
c) an output for releasing the data indicative of the action for causing the change in the likelihood of the certain outcome.

2. An apparatus as defined in claim 1, wherein the health characteristic is modifiable.

3. An apparatus as defined in claim 1, wherein the data derived by the processing unit is indicative of an action for causing the health characteristic to be modified such as to cause the change in the likelihood of the certain outcome.

4. An apparatus as defined in claim 1, wherein the measurement of the health characteristic of the obstetrics patient is indicative of a measurement of a health characteristic of a fetus.

5. An apparatus as defined in claim 1, wherein the measurement of the health characteristic of the obstetrics patient is indicative of a measurement of a health characteristic of a pregnant woman.

6. An apparatus as defined in claim 2, wherein said processing unit is adapted for:
a) processing the signal indicative of a measurement of a health characteristic to derive a data element indicative of the likelihood of the certain outcome;
b) deriving data indicative of an action for causing the health characteristic to be modified such as to cause a change in the likelihood of the certain outcome at least in part on the basis of:
i. the data element indicative of the likelihood of the certain outcome;
ii. the signal indicative of the measurement of the health characteristic of the obstetrics patient.

7. An apparatus as defined in claim 6, wherein the measurement of the health characteristic is indicative of a measurement of a uterine cervix.

8. An apparatus as defined in claim 7, wherein the measurement of the uterine cervix is a measurement of the ripeness of the cervix, said certain outcome being indicative of a delivery by cesarean section.

9. An apparatus as defined in claim 8, wherein the data indicative of the action for causing the health characteristic to be modified is indicative of a treatment for affecting the ripeness of the cervix.

10. An apparatus as defined in claim 3, wherein the output releases a signal suitable for displaying on a display screen the data indicative of the action for causing the health characteristic to be modified.

11. An apparatus as defined in claim 3, wherein the output releases a signal suitable for causing a printing device to display in printed format the data indicative of the action for causing the health characteristic to be modified.

12. An apparatus as defined in claim 6, wherein the measurement of the health characteristic is indicative of the patient's blood type.

13. An apparatus as defined in claim 6, wherein the measurement of the health characteristic is indicative of the patient's disease status.

14. An apparatus as defined in claim 6, wherein the measurement of the health characteristic is indicative of a measurement of the patient's body temperature.

15. A method for monitoring an obstetrics patient, said method comprising:
a) receiving a signal indicative of a measurement of a health characteristic of the obstetrics patient, the measurement being associated with a likelihood of a certain outcome;
b) processing said signal to derive data indicative of an action for causing a change in the likelihood of the certain outcome;
c) releasing the data indicative of the action for causing the change in the likelihood of the certain outcome.

16. A method as defined in claim 15, wherein the health characteristic is modifiable.

17. A method as defined in claim 15, wherein said data is indicative of an action for causing the health characteristic to be modified such as to cause the change in the likelihood of the certain outcome.

18. A method as defined in claim 15, wherein the measurement of the health characteristic of the obstetrics patient is indicative of a measurement of a health characteristic of a fetus.

19. A method as defined in claim 15, wherein the measurement of the health characteristic of the obstetrics patient is indicative of a measurement of a health characteristic of a pregnant woman.

20. A method as defined in claim 15, further comprising:
a) processing said signal indicative of a measurement of a health characteristic to derive a data element indicative of the likelihood of the certain outcome;
b) deriving data indicative of an action for causing the health characteristic to be modified such as to cause a change in the likelihood of the certain outcome at least in part on the basis of:
i. the data element indicative of the likelihood of the certain outcome;
ii. the signal indicative of the measurement of the health characteristic of the obstetrics patient.

21. A method as defined in claim 20, wherein the measurement of the health characteristic is indicative of a measurement of a uterine cervix.

22. A method as defined in claim 20, wherein the measurement of the uterine cervix is a measurement of the ripeness of the uterine cervix, the certain outcome being indicative of a delivery by cesarean section.

23. A method as defined in claim 22, wherein the data indicative of the action for causing the health characteristic to be modified is indicative of a treatment for affecting the ripeness of the cervix.

24. A method as defined in claim 17, comprising releasing a signal suitable for causing a display screen to display the data indicative of the action for causing the health characteristic to be modified.

25. A method as defined in claim 17, wherein the output releases a signal suitable for causing a printing device to display in printed format the data indicative of the action for causing the health characteristic to be modified.

26. An apparatus as defined in claim 20, wherein the measurement of the health characteristic is indicative of the patient's blood type.

27. An apparatus as defined in claim 20, wherein the measurement of the health characteristic is indicative of the patient's disease status.

28. An apparatus as defined in claim 20, wherein the measurement of the health characteristic is indicative of a measurement of the patient's body temperature.

29. A computer readable storage medium including a program element suitable for execution by a computing apparatus, said computing apparatus comprising a memory unit and a processor, said program element when executing on said processor being adapted for:
a) processing a data element indicative of an action for causing a health characteristic of an obstetrics patient to be modified, the data element being generated according to the method described in claim 15;
b) conveying information to a user on either one of a display screen or in paper format, the information being derived at least in part on the basis of the data element processed in a) and being indicative of the action for causing the likelihood of a certain characteristic to be modified.

30. A computer readable storage medium including a program element suitable for execution by a computing apparatus for monitoring an obstetrics patient, said computing apparatus comprising:
a) a memory unit;
b) a processor operatively connected to said memory unit, said program element when executing on said processor being operative for:
i. receiving a signal indicative of a measurement of a health characteristic of the obstetrics patient, the measurement being associated with a likelihood of a certain outcome;
ii. processing said signal to derive data indicative of an action for causing a change in the likelihood of the certain outcome;
iii. releasing the data indicative of the action for causing the likelihood of the certain outcome to be modified.

31. A computer readable storage medium as defined in claim 30, wherein said data is indicative of an action for causing the health characteristic to be modified such as to cause the change in the likelihood of the certain outcome.

32. A computer readable storage medium as defined in claim 30, wherein the measurement of the health characteristic of the obstetrics patient is indicative of a measurement of a health characteristic of a fetus.

33. A computer readable storage medium as defined in claim 30, wherein the measurement of the health characteristic of the obstetrics patient is indicative of a measurement of a health characteristic of a pregnant woman.

34. A computer readable storage medium as defined in claim 31, said program element when executing on said processor being operative for:
a) processing said signal to derive a data element indicative of the likelihood of the certain outcome;
b) deriving data indicative of an action for causing the health characteristic to be modified such as to cause a change in the likelihood of the certain outcome at least in part on the basis of:
i. the data element indicative of the likelihood of the certain outcome;
ii. the signal indicative of the measurement of the health characteristic of the obstetrics patient.

35. A computer readable storage medium as defined in claim 34, wherein the measurement of the health characteristic is indicative of a measurement of a uterine cervix.

36. A computer readable storage medium as defined in claim 34, wherein the measurement of the uterine cervix is a measurement of the ripeness of the uterine cervix, said certain outcome being indicative of a delivery by cesarean section.

37. A computer readable storage medium as defined in claim 36, wherein the data indicative of the action for causing the health characteristic to be modified is indicative of a treatment for modifying the ripeness of the cervix.

38. A computer readable storage medium as defined in claim 31, wherein said program element when executing on said processor is operative for: releasing a signal suitable for causing a display screen to display the data indicative of an action, the action causing the health characteristic to be modified.

39. A computer readable storage medium as defined in claim 31, wherein said program element when executing on said processor is operative for releasing a signal suitable for causing a printing device to display in printed format the data indicative of an action for causing the health characteristic to be modified.

40. A system for monitoring an obstetrics patient, comprising:
a) an interface for receiving a measurement of a health characteristic of the obstetrics patient, the measurement being associated with a likelihood of a certain outcome;
b) an apparatus comprising:
i. an input for receiving a signal indicative of the measurement of the health characteristic of the obstetrics patient;
ii. a processing unit coupled to said input, said processing unit being operative for processing said signal to derive data indicative of an action for causing a change in the likelihood of the certain outcome;
iii. an output coupled to said processing unit, said output being suitable for releasing an output signal for causing a display unit to display information derived on the basis of the data indicative of the action for causing the likelihood of the certain outcome to be modified;
c) a display unit coupled to the output of said apparatus, said display unit being responsive to the output signal to display the information derived on the basis of the data indicative of the action for causing the likelihood of the certain outcome to be modified.

41. A system as defined in claim 40, wherein the interface could be a user interface selected from a keyboard, a pointing device, a touch sensitive screen, and a voice recognition unit.

42. A system as defined in claim 40, wherein said display unit is either one of a display screen and a printing device.

43. A server system for monitoring an obstetrics patient, said server system storing a program element for execution by a CPU, said program element comprising:
a) a first program element component for receiving a measurement of a health characteristic of the obstetrics patient, the measurement being associated with a likelihood of a certain outcome;
b) a second program element component for processing said measurement to derive data indicative of an action for causing a change in the likelihood of the certain outcome;
c) a third program element component for causing the data indicative of the action to be conveyed to a user.

44. A client-server system for monitoring an obstetrics patient, said client-server system comprising a client system and a server system, said client system and said server system operative to exchange messages over a data network, said server system storing a program element for execution by a CPU, said program element comprising:
a) a first program element component executed on said server system for receiving a measurement of a health characteristic of the obstetrics patient, the measurement being associated with a likelihood of a certain outcome;
b) a second program element component executed on said server system for processing said measurement to derive data indicative of an action for causing a change in the likelihood of the certain outcome;
c) a third program element component executed on said server system for sending messages to said client system for causing said client system to display information on the basis of the data indicative of the action for causing the likelihood of the certain outcome to be modified;
d) a fourth program element component executed on said client server system for receiving a message from said server system for displaying the information to a user.

45. A client-server system as defined in claim 44, wherein said fourth program element is adapted for causing the information to be displayed on a display screen.

46. A client-server system as defined in claim 44, wherein said fourth program element is adapted for causing the information to be displayed on a printed medium.

47. A client-server system as defined in claim 44, wherein the data network is the Internet.

48. An apparatus for monitoring an obstetrics patient, said apparatus comprising:
a) means for receiving a signal indicative of a measurement of a health characteristic of the obstetrics patient, the measurement being associated with a likelihood of a certain outcome;
b) means for processing said signal to derive data indicative of an action for causing a change in the likelihood of the certain outcome;
c) means for releasing the data indicative of the action for causing the likelihood of the certain characteristic to be modified.
